(19) United States
(12) Patent Application Publication
(10) Pub. No.: US 2025/0295424 A1
(43) Pub. Date: Sep. 25, 2025

(54) TENACULUM WITH ANTI-SPLAY WING

(71) Applicant: Gynex Corporation, North Richland Hills, TX (US)

(72) Inventors: Trevor R. Kurz, Keller, TX (US); Stephen J. Sullivan, Colleyville, TX (US)

(21) Appl. No.: 19/086,682

(22) Filed: Mar. 21, 2025

Related U.S. Application Data
(60) Provisional application No. 63/568,770, filed on Mar. 22, 2024.

(51) Int. Cl. A61B 17/28

(52) U.S. Cl. CPC A61B 17/2833

(57) ABSTRACT

A surgical instrument that includes a first arm pivotably connected to a second arm at a pivot. The first arm and the second arm form a body having a proximal end and a distal end. A handle is disposed at the proximal end and a grasping tip at the distal end. The first arm includes a receiving body and the second arm includes an anti-splay wing. Slidable engagement of the anti-splay wing and the receiving body maintains alignment of the first arm and the second arm as the surgical instrument transitions between an open configuration and a closed configuration. An end of the anti-splay wing is substantially contained by the receiving body when the surgical instrument is in the closed configuration.

### RELATED APPLICATION

The present application claims priority to U.S. Provisional Patent Application No. 63/568,770, filed Mar. 22, 2024, entitled “Tenaculum with Anti-Sply Wing,” the entirety of which is hereby incorporated by reference for all purposes.

### TECHNICAL FIELD

The present disclosure relates to the field of surgical instruments, and more particularly to forceps with unobtrusive alignment features, such as a tenaculum with an improved anti-splay wing and receiving body.

### BACKGROUND

A tenaculum is a handheld, surgical instrument that is generally classified as a type of forceps. Tenaculums are frequently used during gynecological procedures, and for holding arteries in more general surgical procedures. Non-disposable tenaculums can be made from high-grade carbon steel, stainless steel, or alloys, such as chromium and vanadium. Disposable tenaculums can be made from relatively less expensive, lower-quality materials, such as plastic. Given sufficient force exerted on the handles of the tenaculum can result in flexing during use, causing misalignment of its arms and its grasping tip, making it difficult if not impossible to perform surgical procedures. The disposable tenaculums are particularly susceptible to flexing during use.

### SUMMARY

Novel aspects of the present disclosure are directed to a surgical instrument that includes a first arm pivotably connected to a second arm at a pivot. The first arm and the second arm form a distal portion of a body of the instrument. A first shank is connected to a second shank at the pivot, wherein the first shank and the second shank form a proximal portion of the body. The surgical instrument has a handle at an end of the proximal portion and a grasping tip at an end of the distal portion. Further, the first shack includes a receiving body and the second shack includes an anti-splay wing at the grasping tip of the surgical instrument. Slidable engagement of the anti-splay wing and the receiving body maintains alignment of the first arm and the second arm as the surgical instrument transitions between an open configuration and a closed configuration, and at least an end of the anti-splay wing is substantially contained by the receiving body when the surgical instrument is in the closed configuration.

Other novel aspects of the present disclosure are directed to a surgical instrument that includes a first arm pivotably connected to a second arm at a pivot. The first arm and the second arm form a distal end of a body having a grasping tip. The surgical instrument also includes an alignment feature coupling the first arm with the second arm. The alignment feature maintains alignment of the first arm and the second arm as the surgical instrument transitions between an open configuration and a closed configuration, and the alignment feature does not project outwardly from an outside surface of either the first arm or the second arm.

Other aspects, embodiments and features of the present disclosure will become apparent from the following detailed description of the present disclosure when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the present disclosure shown where illustration is not necessary to allow those of ordinary skill in the art to understand the present disclosure.

### BRIEF DESCRIPTION OF THE DRAWINGS

The novel features believed characteristic of the disclosure are set forth in the appended claims. The novel features, however, as well as a preferred mode of use, further objectives and advantages thereof, will be best understood by reference to the following detailed description of illustrative embodiments when read in conjunction with the accompanying figures, wherein:

FIG. 1 depicts a conventional surgical instrument.

FIGS. 2-8 are various views of the disclosed improved surgical instrument in a closed configuration according to an illustrative embodiment;

FIG. 9 is a cross-sectional view of the disclosed improved surgical instrument in the closed configuration according to an illustrative embodiment;

FIG. 10 is a plan view of a side of the disclosed improved surgical instrument in an open configuration according to an illustrative embodiment;

FIG. 11 is a cross-sectional view of the disclosed improved surgical instrument in an open configuration according to an illustrative embodiment;

FIG. 12 is another cross-sectional view of the disclosed improved surgical instrument illustrated in FIG. 2 in accordance with an illustrative embodiment;

FIGS. 13 and 14 are perspective views of the disclosed improved surgical instrument in a partially separated configuration in accordance with an illustrative embodiment;

FIG. 15 is a perspective view of another embodiment of the disclosed improved surgical instrument in a partially separated configuration; and

FIG. 16 is a cross-sectional view of the disclosed improved surgical instrument illustrated in FIG. 15.

### DETAILED DESCRIPTION

Forceps can be used to grasp and hold objects. Tenaculums are types of forceps that are most commonly used in gynecological procedures. Early versions of forceps, tenaculums in particular, lacked alignment features and were susceptible to flexing (i.e., lateral movement) of its arms during use. The flexing resulted in misalignment of the distal or grasping end of the tenaculum, i.e., misalignment of the shaft and tip. This misalignment prevented the tenaculum from properly grasping and holding objects. To address this problem, later versions of conventional tenaculums were designed to include an excessively long, curved anti-splay wing that allowed the arms of the tenaculum to open and close, but prevented or at least reduced the degree of flexing. However, the length of the anti-splay wing extending out from the outside surface(s) of the arms of conventional tenaculums rendered them obtrusive to the physician/user and the patient.

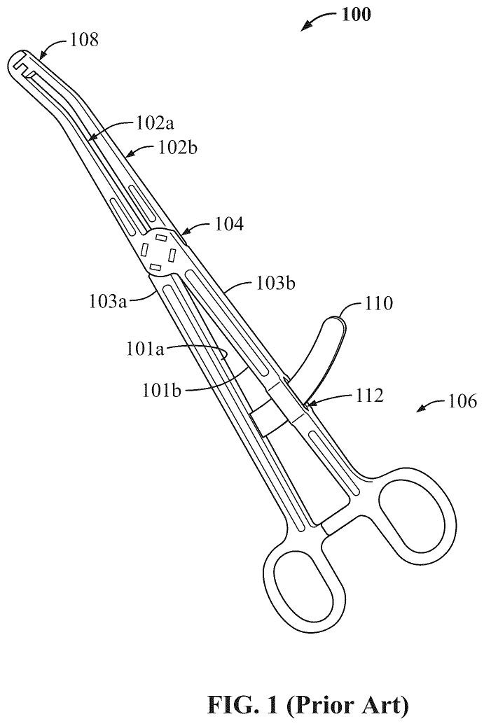

FIG. 1 depicts a conventional forceps for intrauterine device (IUD) removal. The forceps 100 are generally formed from a first arm 102a and a second arm 102b (collectively, “arms 102”) connected at a pivot 104. A user manipulating the handles 106 of the forceps 100 in a manner known by those having ordinary skill in the art can cause the tip 108 of the forceps 100 to open and close, which allows the user to grasp and hold objects via the lever principle.

Each of the arms 102a and 102b can be generally described as an elongated member having an inside surface and an outside surface. The inside surface of each arm 102a and 102b, i.e., inside surface 101a and 101b, faces each other when the arms 102 are assembled and ready for use. The outside surface of each arm 102a and 102b, i.e., outside surface 103a and 103b, face away from each other when the arms 102 are assembled and ready for use.

The forceps 100 include an anti-splay wing 110 that extends outwardly from the inside surface of one of the arms, e.g., inside surface 101a, towards the inside surface of the opposing arm, e.g., inside surface 101b. The anti-splay wing 110 is an elongated, curved member with a first flattened side and an opposing second flattened side, both of which share a curved side wall. During use, the anti-splay wing 110 is slidably inserted into a channel 112 passing through the opposing arm to help maintain alignment of the arms 102 as the forceps 100 open and close. The channel 112 has an opening on the inside surface 101b of the arm 102b and another opening on the outside surface 103b to allow the anti-splay wing 110 to pass completely through the opposing arm 102b. The portion of the anti-splay wing 110 extending out past the outer surface 103b of the opposing arm 102b is termed “overhang”. The maximum amount of overhang is encountered when the forceps 100 is in the fully closed position.

The width of channel 112 is dimensioned relative to the width of the anti-splay wing 110 to prevent undesirable lateral movement that would result in misalignment of the arms 102a and 102b and, as a consequence, misalignment of the ends of tip 108. If the arms 102 are considered to define a plane, e.g., the X-Y plane, then the type of lateral movement that can be reduced by the anti-splay wing 110 traveling through the channel 112 is in a direction normal to that plane, i.e., in the ±Z direction.

The length of anti-splay wing 110 generally dictates the degree to which the forceps 100 can be opened during use without the anti-splay wing 110 being withdrawn from the corresponding channel 112. Withdrawal of the anti-splay wing 110 from the channel 112 could prevent the forceps 100 from being closed if the arms 102 become misaligned during use. While a greater length of the anti-splay wing 110 would lessen the chance that the anti-splay wing 110 would be inadvertently withdrawn from the channel 112, the greater the length of the anti-splay wing 110, the greater the degree of overhang and the more obtrusive the anti-splay wing 110 can be for the user and the patient. For example, a user's grip on the forceps 100 may need to be modified as the forceps 100 is closed and the amount of overhang increases. Additionally, initial deployment of the forceps 100 may be prevented if space constraints cannot accommodate such an increased amount of overhang. Further, the overhang of the anti-splay wing 110 can prevent twisting or repositioning of the forceps 100. Novel aspects of this disclosure recognize the need for improved alignment features for forceps and forceps-like devices which are non-obtrusive, or at least less obtrusive to the user and the patient.

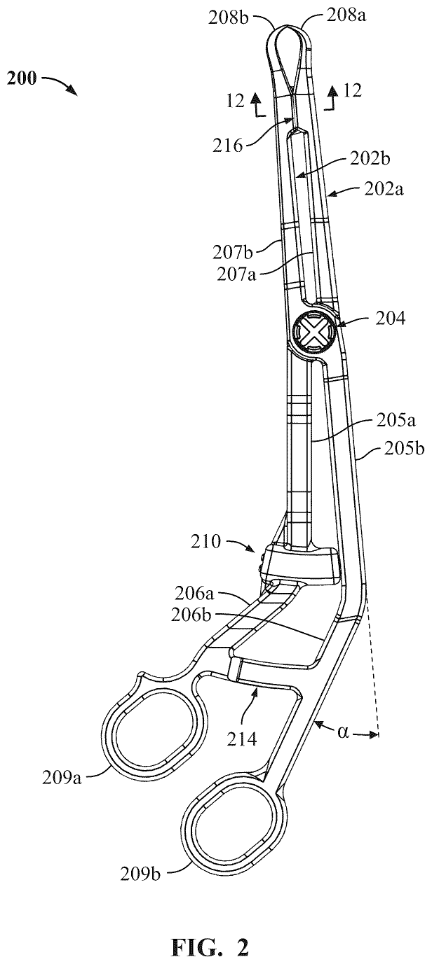

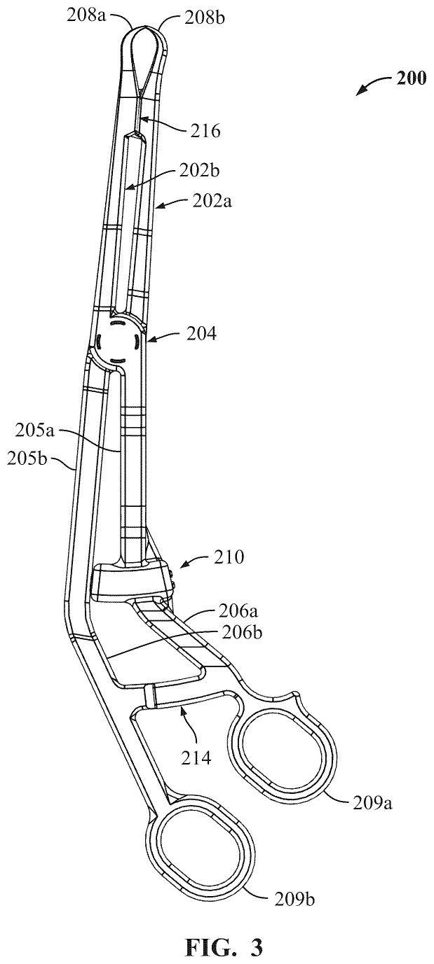

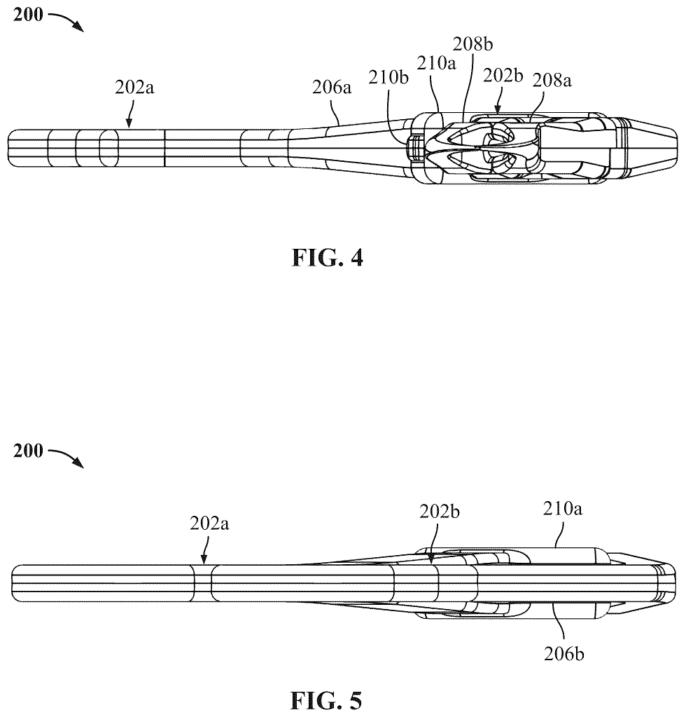

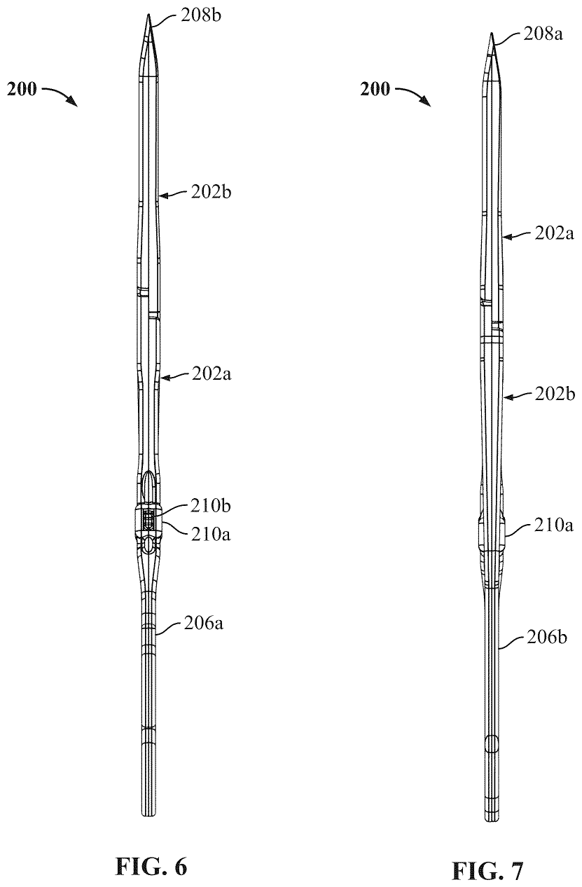

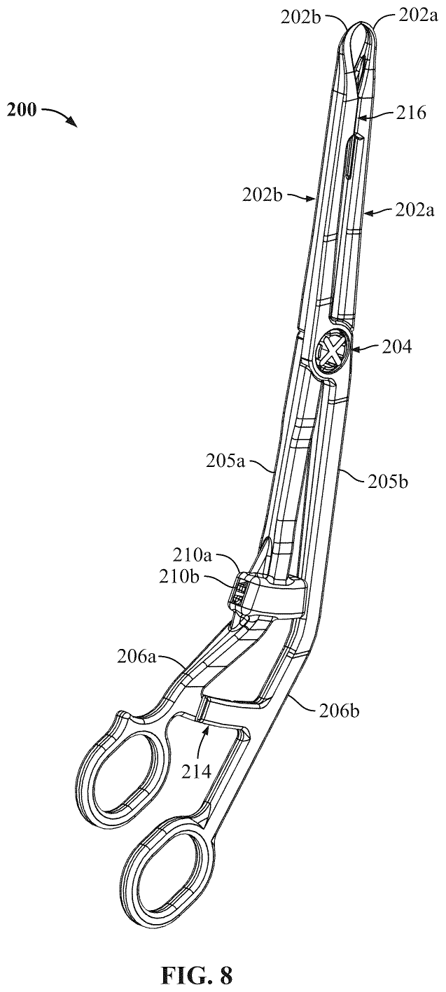

FIGS. 2-7 are various views of the improved surgical instrument in accordance with an illustrative embodiment. The surgical instrument is depicted as a tenaculum, but can be replaced with any one of the many various forceps-like devices. FIGS. 2 and 3 are plan views of a first and second side of the tenaculum 200. FIGS. 4 and 5 are front and rear edge views of the tenaculum 200, and FIGS. 6 and 7 are bottom and top edge views of the tenaculum 200. FIG. 8 is a perspective view of the tenaculum 200.

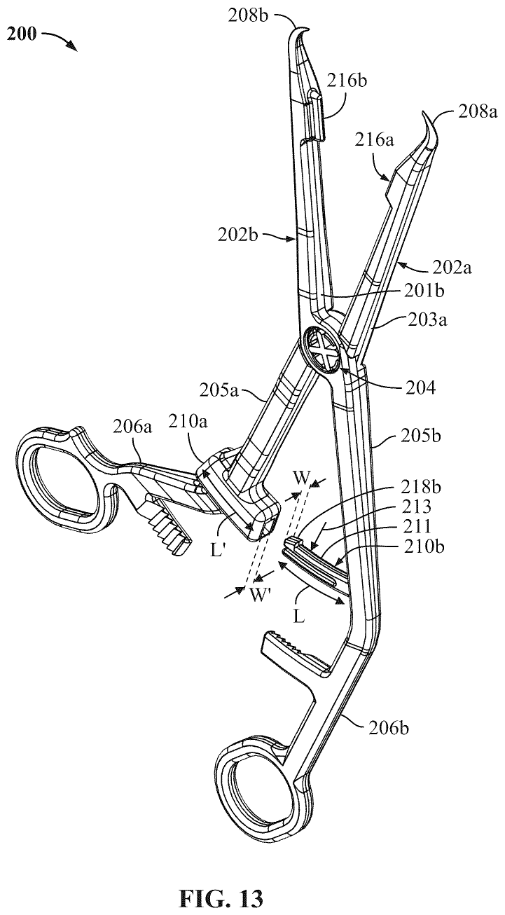

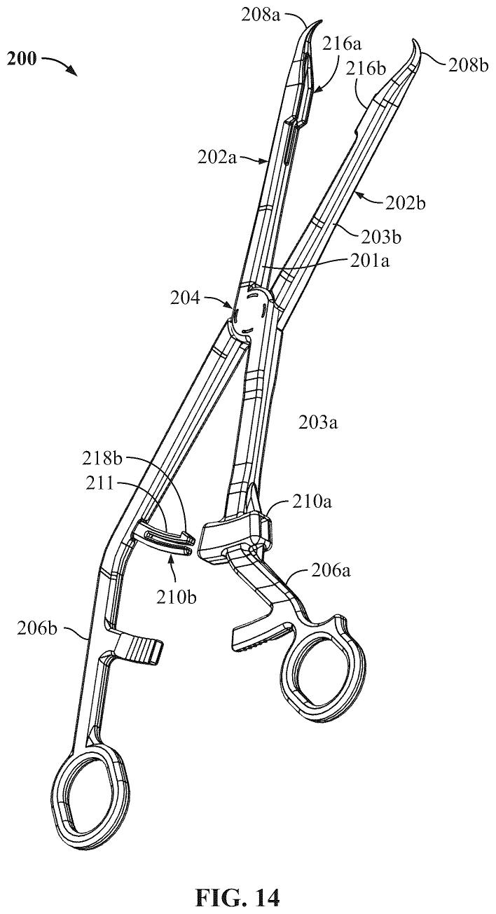

The tenaculum 200 is formed generally from a first arm 202a pivotably connected to a second arm 202b (collectively, arms 202) at pivot 204. The pivot 204 divides the tenaculum 200 generally into a distal portion and a proximal portion. The distal portion includes shafts 207a and 207b (collectively, shaft 207), each of which is connected to a corresponding tip portion 208a and 208b (collectively, tip 208). The proximal portion includes shanks 205a and 205b (collectively, shanks 205), each of which is connected to a corresponding handle portion 206a and 206b (collectively, handle 206). The handle 206 can include a pair of finger rings 209a and 209b (collectively, finger rings 209). Further, each arm 202a and 202b has an inside surface 201a and 201b, and an outside surface 203a and 203b, which are shown in more detail in FIGS. 13 and 14.

To improve user comfort during use, the handle 206 can be angled relative to the shank 205. The angle α, which is measured by determining the angle between one of the shanks 205a or 205b and the attached handle 206a or 206b, can be between 15°-45°, but more particularly about 30°. The angle α provides a more neutral grip in certain medical procedures, such as gynecological procedures given the relative location and positioning of the patient and the physician using the tenaculum 200.

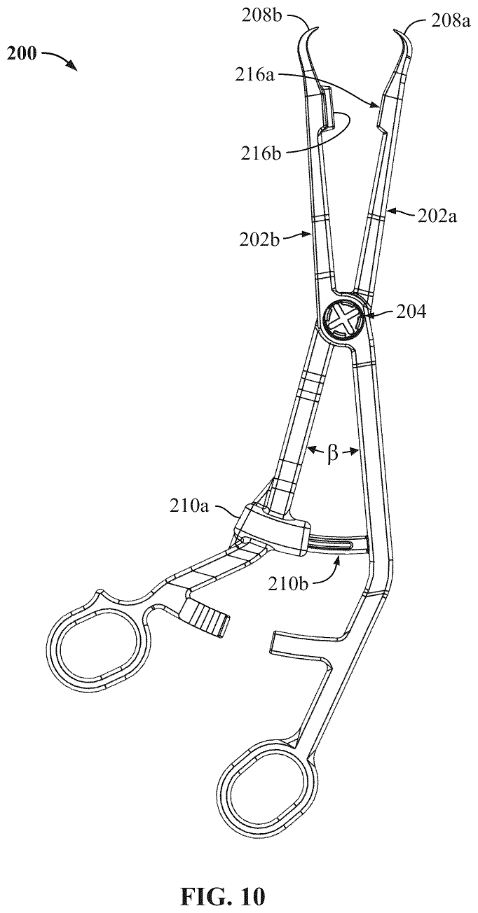

The tenaculum 200 also includes a ratchet 214, which includes interlocking ridges that are shown in more detail in FIGS. 10, 13, and 14. The ratchet 214 allows the tenaculum 200 to maintain the closed configuration without the need for a user's continued application of a clamping force on the handles 206. Thus, a user can grasp an object by the tip 208 of the tenaculum 200 and engage the interlocking ridges of the ratchet 214, which causes the tenaculum 200 to maintain the grasping force on the object until the interlocking ridges are disengaged.

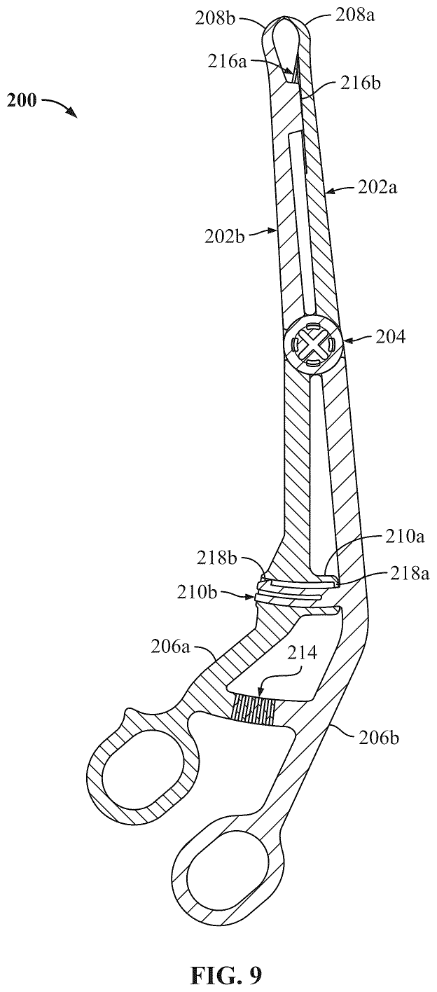

The tenaculum 200 also includes a proximal alignment feature 210 disposed between the pivot 204 and the handle 206, which is configured to help maintain alignment of the arms 202 relative to one another during use. In particular, the alignment feature 210 allows the arms 202 to rotate relative to one another at pivot 204 but prevents or at least reduces the amount of lateral movement of the arms 202 while also eliminating or at least substantially reducing the amount of overhang relative to prior art tenaculums, such as tenaculum 100 in FIG. 1. In this illustrative embodiment, the alignment feature 210 includes an anti-splay wing 210b, shown in more detail in FIGS. 9, 10, 11, 13, and 14, and a receiving body 210a. In the non-limiting embodiment disclosed herein, the first arm 202a includes the receiving body 210a and the second arm 202b includes the anti-splay wing 210b.

The anti-splay wing 210b is an elongated and curved member that is configured to be slidably engaged within the channel 212 of the receiving body 210a. The curvature of the anti-splay wing 210b is the same or similar to the curvature of the channel 212 to allow the anti-splay wing 210b to slide in and out of the channel 212, which in turn allows the arms 202 to open and close. The width of the channel 212 relative to the width of the anti-splay wing 210b controls the lateral movement of the arms 202. For example, with reference to FIG. 13, the anti-splay wing 210b has a width W and the channel 212 in the receiving body 210a has a width W′ that is greater than W. The difference between W′ and W dictates the amount of permissible lateral movement. The smaller the difference between W′ and W, the lesser the amount of lateral movement of the arms 202, but the greater the amount of frictional forces between them. The greater the difference between W′ and W, the greater the amount of lateral movement of the arms 202 and the lesser the amount of frictional forces (if any) between them.

By containing all or at least substantially all of the end portion of the anti-splay wing 210b within the receiving body 210a when the tenaculum 200 is in the closed position, the amount of overhang of the anti-splay wing 210b projecting out of the outside surface 203a of the arm 202a is eliminated, or at least substantially reduced as compared to conventional tenaculums. In the depicted embodiment, the receiving body 210a is shown as a sleeve with a length L′ that is approximately the same as the length L of the anti-splay wing 210b. As a result, the anti-splay wing 210b is entirely or at least almost entirely contained within the receiving body 210a when the tenaculum 200 is in the closed configuration. In other embodiments, the receiving body 210a can be partially opened on one or both sides, as long as the anti-splay wing 210b can be contained within the volume defined by the receiving body 210a while the tenaculum 200 is at least in the closed configuration to prevent undesirable overhang. In any of these embodiments, the alignment feature 210 can also include a slide stop 218, shown in more detail in FIGS. 9, 11, 13, and 14, to prevent the anti-splay wing 210b from being inadvertently withdrawn from the receiving body 210a.

The tenaculum 200 can also include a distal alignment feature 216 disposed between the pivot 204 and the grasping tip 208, i.e., located somewhere along the distal portion of the tenaculum 200. The distal alignment feature 216a and 216b (collectively, 216) is comprised of features 216a and 216b configured to guide respective grasping tips 208a and 208b together as the tenaculum 200 is closed, which prevents or at least reduces the tendency for each of the grasping tips 208a and 208b to experience undesirable lateral movement and overlapping. In an embodiment, no portion of the alignment feature 216 extends out past the outside surfaces 203 of the arms 202 such that the tenaculum 200 has a more streamlined form factor, which promotes ease of use and patient comfort, particularly when implemented in gynecological procedures. In another embodiment, a small portion of the alignment feature 216 may extend out slightly past the outside surfaces 203 of the arms 202 to more effectively prevent or reduce undesirable lateral movement and overlapping of the grasping tips 208a and 208b while still maintaining the streamlined form factor of the tenaculum 200.

The alignment feature 216 depicted in the illustrated embodiment is formed from a ridge 216b and a groove 216a sized to receive the ridge 216b. The groove 216a is defined by a pair of sidewalls extending outwardly from the inside surface 201a of the arm 202a, and the ridge 216b extends outwardly from the inside surface 201b of the opposing arm 202b. The depicted alignment feature 216 should be deemed exemplary and non-limiting. For example, the ridge 216b can be replaced by a smaller anti-splay wing extending from one of the arms 202a or 202b and the groove 216a can be replaced by a receiving body of the opposing arm 202b or 202a, such as a curved channel. By limiting the length of the anti-splay wing or the depth of the channel to prevent the anti-splay wing from extending out past the side of the opposing arm, the streamlined form factor can still be achieved.

FIG. 9 is a cross-sectional view of the improved surgical instrument in accordance with an illustrative embodiment. The cross-sectional view is taken of tenaculum 200 in FIG. 1 along a plane that bisects both the arms 202a and 202b. As can be seen, in the closed configuration, the anti-splay wing 210b is contained in the channel 212 defined by the receiving body 210a, and the ridge 216b is contained in the groove 216a. Because the ridge 216b and the anti-splay wing 210b are contained entirely or at least substantially entirely within groove 216a and receiving body 210a, respectively, the amount of overhang is substantially if not entirely eliminated.

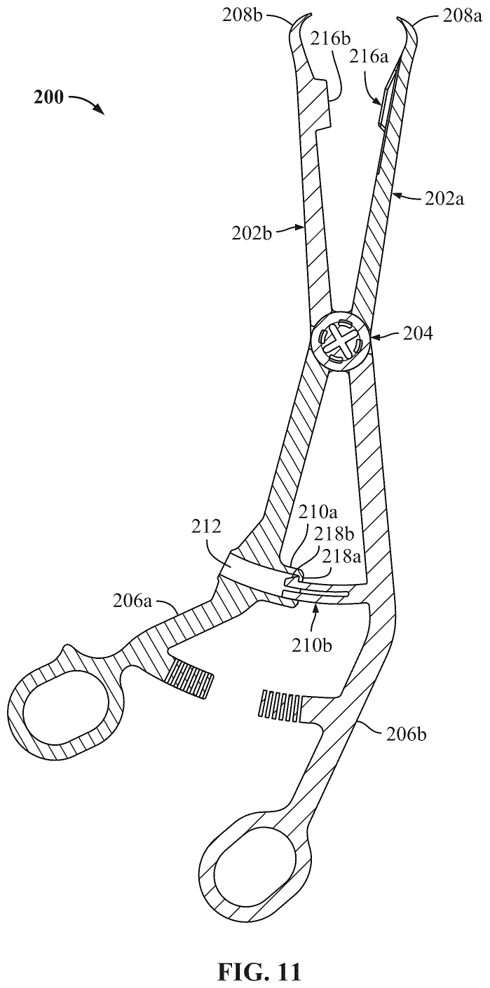

FIGS. 10 and 11 are various views of the improved surgical instrument in an open configuration according to an illustrative embodiment. In particular, FIG. 10 is a plan view of a side of the improved surgical instrument and FIG. 11 is a cross-sectional view of the tenaculum 200 in FIG. 10 and taken along a plane that bisects each of the arms 202a and 202b.

The tenaculum 200 can be opened by manipulating the handles 206 in a manner that is known to those having ordinary skill in the art. The degree to which the arms 202 of the tenaculum 200 can be opened, i.e., the magnitude of the angle β, can be determined, at least in part, by the placement of the slide stop 218, shown in more detail in FIG. 11. In the depicted embodiment, the slide stop 218 is formed from a first locking interface and a second locking interface. The first and second locking interfaces are depicted as a one-way barb 218b and a lip 218a, respectively. The angle β is achieved when the one-way barb 218b and the lip 218a come into contact. Placement of the one-way barb 218b at the end of the anti-splay wing 210b and the lip 218a at the entrance of the channel 212 defined by the receiving body 210a permits the tenaculum 200 to be opened to allow a maximum value of β. Moving the lip 218a further into the channel 212 and/or moving the one-way barb 218b further down the anti-splay wing 210b away from the end of the anti-splay wing 210b reduces the value of β that can be achieved.

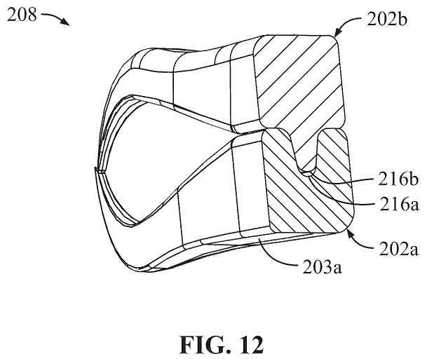

FIG. 12 is another cross-sectional view of the improved surgical instrument in accordance with an illustrative embodiment. The cross-sectional view is taken along lines 12-12 in FIG. 2, which depicts the distal alignment feature 216 in greater detail. The alignment feature 216 in FIG. 12 includes a ridge 216b and groove 216a that is sized to receive the ridge 216b. The ridge 216b is disposed on arm 202b, projecting toward the groove 216a disposed on the opposing arm 202a. As the tenaculum 200 transitions to the closed configuration, the ridge 216b is slidably inserted into the groove 216a, which brings the distal end of the arms 202, and thus the tip portions 208, into alignment. Because the ridge 216b does not project out past the outside surface 203a of the arm 202a, the streamlined form factor is preserved. With some conventional tenaculums, the two tip portions often include alignment features that align the tip portions as they come together; however, providing such an alignment feature in the tip of such an instrument can interfere with the grasp provided by the tip portions. Thus, in embodiments according to the disclosed principles, by providing this second alignment feature to the alignment provided by the anti-splay wing 210b and channel 212 away from the tip 208 of the instrument, the grasp provided by the instrument is not affected.

FIGS. 13 and 14 are perspective views of the improved surgical instrument in a partially separated configuration in accordance with an illustrative embodiment. The partially separated configuration depicts the arms 202 of the tenaculum 200 separated far enough apart such that the anti-splay wing 210b and the receiving body 210a are disengaged from one another. The partially separated configuration is generally encountered as an intermediate step during assembly of the tenaculum 200. For example, manufacture of the tenaculum can involve forming the first arm 202a and the second arm 202b separately, then assembling the tenaculum 200 by first coupling the arms 202 at the pivot 204, which achieves the partially separated configuration shown in FIGS. 13 and 14. A closing force applied to the handles 206 causes the anti-splay wing 210b to mate with the receiving body 210a. As the anti-splay wing 210b enters the opening of receiving body 210a, the lip 218a of the receiving body 210a engages the one-way barb 218b, which causes the prong 211 of the anti-splay wing 210b to flex. Flexing permits insertion of the anti-splay wing 210b into the receiving body 210a. Thereafter, a pressing force applied to the prong 211 in the direction of arrow 213 can cause the requisite flexing to allow the anti-splay wing 210b to be withdrawn from the receiving body 210a.

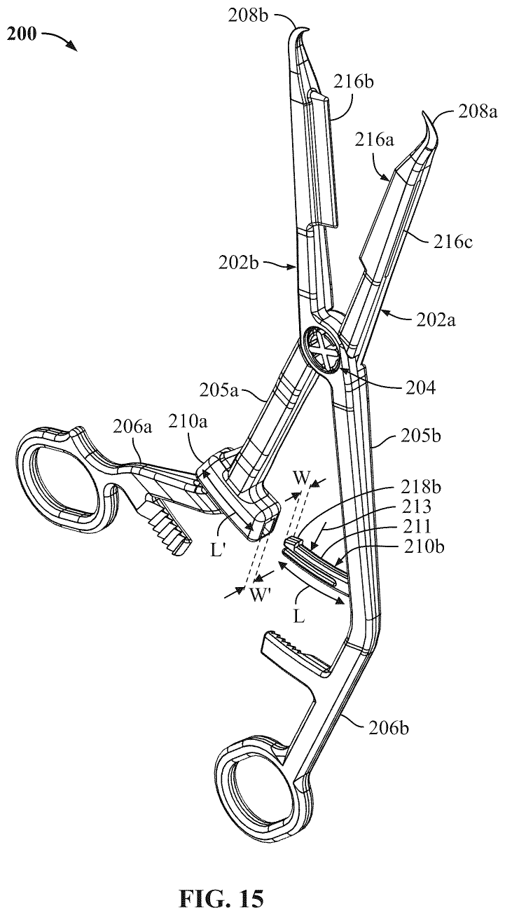

FIG. 15 is a perspective view of the improved surgical instrument in a partially separated configuration in accordance with another illustrative embodiment. As previously mentioned, the alignment feature 216 prevents or at least reduces the tendency for each of the grasping tips 208a and 208b to experience undesirable lateral movement and overlapping when the tenaculum 200 is in the closed configuration. In the non-limiting exemplary embodiment illustrated in FIG. 15, the alignment feature 216 may extend along a greater length of the inside surfaces 201 of the arms 202 as compared to the alignment feature 216 of FIG. 14, for example. More specifically, the ridge 216b may extend along a greater length of the inside surface 201b of arm 202b and the sidewalls forming the groove 216a may extend along a greater length of the inside surface 201a of arm 202a. In some embodiments, the length of one or both of the sidewalls forming the groover 216a and the ridge 216b can extend along more than half of the lengths of the arms 202. Additionally, the ridge 216b and the sidewalls forming the groove 216a may extend a greater outward distance from the inside surfaces 201 of the arms 202. The groove 216a may also extend through arm 202a to form a slot 216c disposed through arm 202a and the outside surface 203a of arm 202a. The slot 216c may be sized to receive the ridge 216b. By increasing the length and depth of the alignment feature 216, the overlapping surface area of the ridge 216b and groove 216a may be increased such that the alignment feature 216 may more effectively prevent or reduce undesirable lateral movement and overlapping of the grasping tips 208a and 208b when the tenaculum 200 is in the closed configuration. In some embodiments having the slot 216c, the tips 208a, 208b may be configured to overlap a predetermined amount when the ridge 216b is received within or passes through the slot 216c. In some embodiments, the slot 216c may be sized so as to provide a friction fit to the ridge 216b, where some resistance to the ridge 216b entering and passing through the slot 216c provides tactile feedback to the user of the instrument to let them know that further closure of the instrument will result in overlapping of the tips 208, 208b.

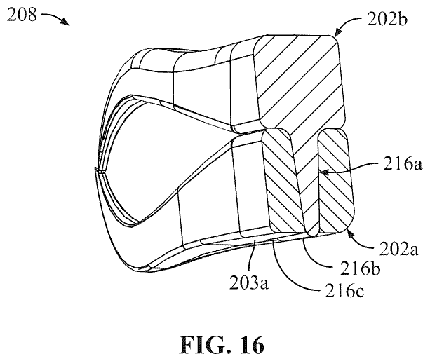

FIG. 16 is cross-sectional view of the improved surgical instrument in accordance with an illustrative embodiment. The cross-sectional view is taken along a line similar to line 12-12 in FIG. 2, but with the embodiment of the instrument illustrated in FIG. 15, and depicts an embodiment of the distal alignment feature 216 in greater detail. As previously mentioned, the alignment feature 216 includes a ridge 216b disposed on arm 202b and a groove 216a sized to receive the ridge 216b and disposed on arm 202a. As the tenaculum 200 transitions to the closed configuration, the ridge 216b is slidably inserted into the groove 216a, which brings the distal end of the arms 202, and thus the tip portions 208, into alignment. In this embodiment, the groove 216a extends through arm 202a to form the slot 216c disposed through arm 202a and the outside surface 203a of arm 202a. In the non-limiting exemplary embodiment illustrated in FIG. 16, the ridge 216b may project through the slot 216c out slightly past the outside surface 203a of the arm 202a when the tenaculum 200 is in the closed configuration. With the ridge 216b extending through arm 202a and out slightly past the outside surfaces 203 of arm 202a, alignment feature 216 may more effectively prevent or reduce undesirable lateral movement and overlapping of the grasping tips (not shown) while still maintaining the streamlined form factor of the tenaculum 200. In some embodiments, the groove 216a, ridge 216b, and slot 216c are all correspondingly tapered such that the alignment of the tips 208a, 208b during closure of the instrument increases as it is closed.

Although embodiments of the disclosure have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While the novel aspects of the present disclosure have been particularly shown and described with reference to preferred embodiments, it will be understood by those skilled in the art that various changes in form and detail may be made therein without departing from the spirit and scope of the disclosure. The inventors expect skilled artisans to employ such variations as appropriate, and the inventors intend the novel aspects to be practiced otherwise than as specifically described herein. For example, the tip 208 is depicted as a single-tooth tip, but the tip 208 can be replaced by any one or more conventional tips, including but not limited to sponge forceps, vulsellum, IUD grasper, IUD string trimmer, and IUD string scissor tips. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

We claim:

1. A surgical instrument comprising:
a first arm pivotably connected to a second arm at a pivot, wherein the first arm and the second arm form a distal portion;
a first shank connected to a second shank at the pivot, wherein the first shank and the second shank form a proximal portion;
a handle at an end of the proximal portion; and
a grasping tip at an end of the distal portion, wherein:
the first shank includes a receiving body,
the second shank includes an anti-splay wing,
slidable engagement of the anti-splay wing and the receiving body maintains alignment of the first arm and the second arm as the surgical instrument transitions between an open configuration and a closed configuration, and
at least an end of the anti-splay wing is substantially contained by the receiving body when the surgical instrument is in the closed configuration.

2. The surgical instrument of claim 1, wherein the end of the anti-splay wing is substantially contained by the receiving body when the surgical instrument is in the open configuration.

3. The surgical instrument of claim 1, wherein:
the receiving body is an at least partially closed sleeve that defines a curved channel, and
engagement of the anti-splay wing with one or more sidewalls of the curved channel maintains the alignment of the first arm and the second arm as the surgical instrument transitions between the open configuration and the closed configuration.

4. The surgical instrument of claim 1, wherein:
the receiving body has a length that is substantially the same as a length of the anti-splay wing; and
the receiving body has a curvature that is substantially the same as a curvature of the anti-splay wing.

5. The surgical instrument of claim 1, wherein:
the anti-splay wing includes a first locking interface,
the receiving body includes a second locking interface configured to engage the first locking interface,
engagement of the first locking interface with the second locking interface prevents the anti-splay wing from being withdrawn from the receiving body.

6. The surgical instrument of claim 5, wherein:
the first locking interface is a one-way barb disposed at the end of the anti-splay wing,
the second locking interface is a lip that narrows an entrance of the receiving body, and
the one-way barb permits insertion of the anti-splay wing into the entrance of the receiving body but prevents inadvertent withdrawal of the anti-splay wing from the entrance of the receiving body.

7. The surgical instrument of claim 6, wherein:
the one-way barb is disposed on a flexible bar that is movable to permit disengagement of the one-way barb from the lip to permit withdrawal of the anti-splay wing from the receiving body.

8. The surgical instrument of claim 1, further comprising:
a ratchet disposed on one or more of the first and second shanks, and configured to secure the first arm with the second arm in the closed configuration.

9. The surgical instrument of claim 1, further comprising:
an alignment feature disposed on the first and second arms between the pivot and the grasping tip, wherein the alignment feature is completely contained on opposing inside surfaces of the first and second arms.

10. The surgical instrument of claim 9, wherein:
the alignment feature comprises a ridge on one of the first or the second arm and a groove on the other of the first or second arm,
the groove is sized to receive the ridge, and
the groove receiving the ridge maintains the alignment of the first arm and the second arm as the surgical instrument transitions to the closed configuration.

11. The surgical instrument of claim 1, wherein the surgical instrument is formed from a polymer.

12. The surgical instrument of claim 1, wherein the handle deviates from the first and second shanks by an angle of departure that is between 15°-45°.

13. The surgical instrument of claim 12, wherein the angle of departure is about 30°.

14. A surgical instrument comprising:
a first arm pivotably connected to a second arm at a pivot, wherein the first arm and the second arm form a distal portion of a body;
a first shank connected to a second shank at the pivot, wherein the first shank and the second shank form a proximal portion of the body;
wherein the proximal end includes a handle and the distal end including a grasping tip; and
an alignment feature coupling the first shank with the second shank, wherein the alignment feature maintains alignment of the first arm and the second arm as the surgical instrument transitions between an open configuration and a closed configuration, and wherein the alignment feature is contained on opposing inside surfaces of either the first shank or the second shank.

15. The surgical instrument of claim 14, wherein:
the alignment feature further comprises an anti-splay wing and a receiving body,
the first shank includes the anti-splay wing, and
the second shank includes the receiving body.

16. The surgical instrument of claim 15, wherein:
the anti-splay wing includes a first locking interface,
the receiving body includes a second locking interface configured to engage the first locking interface,
engagement of the first locking interface with the second locking interface prevents the anti-splay wing from being withdrawn from the receiving body.

17. The surgical instrument of claim 16, wherein:
the first locking interface is a one-way barb disposed at the end of the anti-splay wing,
the second locking interface is a lip that narrows an entrance of the receiving body, and
the one-way barb permits insertion of the anti-splay wing into the entrance of the receiving body but prevents inadvertent withdrawal of the anti-splay wing from the entrance of the receiving body.

18. The surgical instrument of claim 17, wherein:
the one-way barb is disposed on a flexible bar that is movable to permit disengagement of the one-way barb from the lip to permit withdrawal of the anti-splay wing from the receiving body.

19. The surgical instrument of claim 14, further comprising:
a ratchet disposed on one or more of the first and second shanks, and configured to secure the first arm with the second arm in the closed configuration.

20. The surgical instrument of claim 14, further comprising:
an alignment feature disposed on the first and second arms between the pivot and the grasping tip, wherein the alignment feature is completely contained on opposing inside surfaces of the first and second arms.
